(19) United States
(12) Patent Application Publication
(10) Pub. No.: US 2020/0261057 A1
(43) Pub. Date: Aug. 20, 2020

(54) SIZE ADJUSTABLE ULTRASOUND ATTACHMENT SYSTEM

(71) Applicant: Luiz MARACAJA, San Antonio, TX (US)

(72) Inventor: Luiz MARACAJA, San Antonio, TX (US)

(73) Assignee: THE BOARD OF REGENTS OF THE UNIVERSITY OF TEXAS SYSTEM, Austin, TX (US)

(21) Appl. No.: 16/753,112

(22) PCT Filed: Oct. 2, 2018
(86) PCT No.: PCT/US2018/053896

§ 371 Date: Apr. 2, 2020

Related U.S. Application Data
(60) Provisional application No. 62/566,629, filed on Oct. 2, 2017.
(60) Provisional application No. 62/593,698, filed on Dec. 1, 2017.

(51) Int. Cl. A61B 8/00; A61B 8/06; A61B 18/14

(52) U.S. Cl. CPC A61B 8/4411; A61B 8/06; A61B 2018/00595; A61B 18/1402; A61B 8/58

(57) ABSTRACT

Embodiments are directed to an ultrasound attachment that flexible interacts with an ultrasound probe and provides for securing the attachment to the probe.

### CROSS REFERENCE TO RELATED APPLICATIONS

This application claims priority to U.S. Provisional Applications 62/566,629 filed Oct. 2, 2017 and 62/593,698 filed Dec. 1, 2017, each of which is incorporated herein by reference in its entirety.

### STATEMENT REGARDING FEDERALLY FUNDED RESEARCH

None.

### BACKGROUND OF THE INVENTION

### A. Field of the Invention

This disclosure is related to medicine in the field of ultrasound guided procedures and intra-operative ultrasound. Ultrasound is part of current clinical practice and for several different purposes such as diagnostic, ultrasound guidance procedures, surgical planning, identification of tumor margins, identification of aortic atheromatous disease, identification of vessels, nerves and other anatomical or pathologic structures.

### B. Description of Related Art

In order to obtain imaging and visualization of the needle when performing ultrasound guided procedures, precise alignment between the needle inserted and ultrasound beam emanated from the probe is necessary, different needle guide systems have been developed. The attachment between the probe and the needle guide is shape and size specific. Since ultrasound probe from different manufacturers vary in shape and size. The probe attachments have to be specifically designed to fit over a specific ultrasound probe.

### SUMMARY OF THE INVENTION

Described herein is an ultrasound attachment system for an ultrasound transducer that accounts for changes in probe shape and size. The system allows attachment and stabilization of different accessories to ultrasound probes from different manufacturers, that is the attachment system is a universal system and is not manufacturer specific. The attachment system has two or more living hinges and two or more stabilizing hooks positioned on each side of the probe. The attachment can have a bottom configured for interaction with an ultrasound target (e.g., a patient), an open top configured to receive an ultrasound probe, two long sides forming, with the bottom portion a U shaped attachment (in certain aspects the attachment can include two additional short sides configured to flexibly interact with a probe as well), the sides configured to flexibly interact with the ultrasound probe and provide for securing the attachment to a probe and/or a probe cover. In certain aspects the probe and/or probe cover can be secured using a flexible band that can be wrapped around the probe and a probe cover with the band being engaged with the stabilizing hooks of the attachment resulting in an operable probe/attachment configuration. In other aspects, the attachment system can hold a standoff chamber that can allow passage of a separate device (e.g. an electrocautery). The tip of the electrocautery can be aligned with the ultrasound transducer and to permit accurate depth adjustment. In certain aspects, the electrocautery tip can have a linear or hockey stick shape to allow surface markings or surgical incisions based on the imaging obtained by the ultrasound transducer.

The living hinges provide size and shape adjustment and account for the variations between different probe manufacturers. A living hinge is a thin flexible hinge (flexure bearing) made from the same material as the two rigid pieces it connects, rather than cloth, leather, or some other flexible substance. It is typically thinned or cut to allow the rigid pieces to bend along the line of the hinge.

When performing ultrasound in the sterile field, a sterile cover is used over the ultrasound transducer and bands (e.g., rubber or other elastic type of bands) are normally used to attach and stabilize the cover over the probe. After the cover and rubber bands are placed over the probe, the attachment system is oriented in the desired position and the rubber bands looped around the stabilizing hooks to securely hold the attachment to the probe.

Other embodiments of the invention are discussed throughout this application. Any embodiment discussed with respect to one aspect of the invention applies to other aspects of the invention as well and vice versa. Each embodiment described herein is understood to be embodiments of the invention that are applicable to all aspects of the invention. It is contemplated that any embodiment discussed herein can be implemented with respect to any method or composition of the invention, and vice versa.

The use of the word “a” or “an” when used in conjunction with the term “comprising” in the claims and/or the specification may mean “one,” but it is also consistent with the meaning of “one or more,” “at least one,” and “one or more than one.”

The term “about” or “approximately” are defined as being close to as understood by one of ordinary skill in the art. In one non-limiting embodiment the terms are defined to be within 10%, preferably within 5%, more preferably within 1%, and most preferably within 0.5%.

The term “substantially” and its variations are defined to include ranges within 10%, within 5%, within 1%, or within 0.5%.

The terms “inhibiting” or “reducing” or “preventing” or any variation of these terms includes any measurable decrease or complete inhibition to achieve a desired result.

The term “effective,” as that term is used in the specification and/or claims, means adequate to accomplish a desired, expected, or intended result.

The terms “wt. %,” “vol. %,” or “mol. %” refers to a weight, volume, or molar percentage of a component, respectively, based on the total weight, the total volume, or the total moles of material that includes the component. In a non-limiting example, 10 moles of component in 100 moles of the material is 10 mol. % of component.

The use of the term “or” in the claims is used to mean “and/or” unless explicitly indicated to refer to alternatives only or the alternatives are mutually exclusive, although the disclosure supports a definition that refers to only alternatives and “and/or.”

As used in this specification and claim(s), the words “comprising” (and any form of comprising, such as “comprise” and “comprises”), “having” (and any form of having, such as “have” and “has”), “including” (and any form of including, such as “includes” and “include”) or “containing” (and any form of containing, such as “contains” and “contain”) are inclusive or open-ended and do not exclude additional, unrecited elements or method steps.

The compositions and methods of making and using the same of the present invention can “comprise,” “consist essentially of,” or “consist of” particular ingredients, components, blends, method steps, etc., disclosed throughout the specification.

Other objects, features and advantages of the present invention will become apparent from the following detailed description. It should be understood, however, that the detailed description and the specific examples, while indicating specific embodiments of the invention, are given by way of illustration only, since various changes and modifications within the spirit and scope of the invention will become apparent to those skilled in the art from this detailed description.

### BRIEF DESCRIPTION OF THE DRAWINGS

The following drawings form part of the present specification and are included to further demonstrate certain aspects of the present invention. The invention may be better understood by reference to one or more of these drawings in combination with the detailed description of the specification embodiments presented herein.

FIG. 1. Is an illustration demonstrating a probe having an attachment secured by the stabilizing hooks and a rubber band.

FIG. 2. Is an illustration of a perspective view of one example of an attachment described herein.

FIG. 3. Is an end view of the attachment of FIG. 2.

FIG. 4. Is a top view of the attachment of FIG. 2.

FIG. 5. Is a side view of the attachment of FIG. 2.

FIG. 6. Is an illustration of the probe attachment incorporating an adjustable caliper.

FIG. 7. Is a perspective illustration of the probe attachment incorporating an adapter that can be configured to allow access for instruments and/or marking pens.

FIG. 8. Is top-view illustration of the probe attachment incorporating an adapter that can be configured to allow access for instruments and/or marking pens.

FIG. 9. Is a side-view illustration of the probe attachment incorporating an adapter that can be configured to allow access for instruments and/or marking pens.

### DETAILED DESCRIPTION OF THE INVENTION

Aspects of the present invention relate to an ultrasound attachment and an ultrasound probe engaged with the ultrasound attachment. The present invention also relates to various ultrasound kits and ultrasound coupling systems configured to include the ultrasound attachment of the present invention.

The ultrasound attachment, devices, and systems of the present invention have various attributes, as described more fully herein. In particular embodiments, the ultrasound attachment is configured for limited use or one-time use. In other aspects the ultrasound attachments can be used, sterilized and reused.

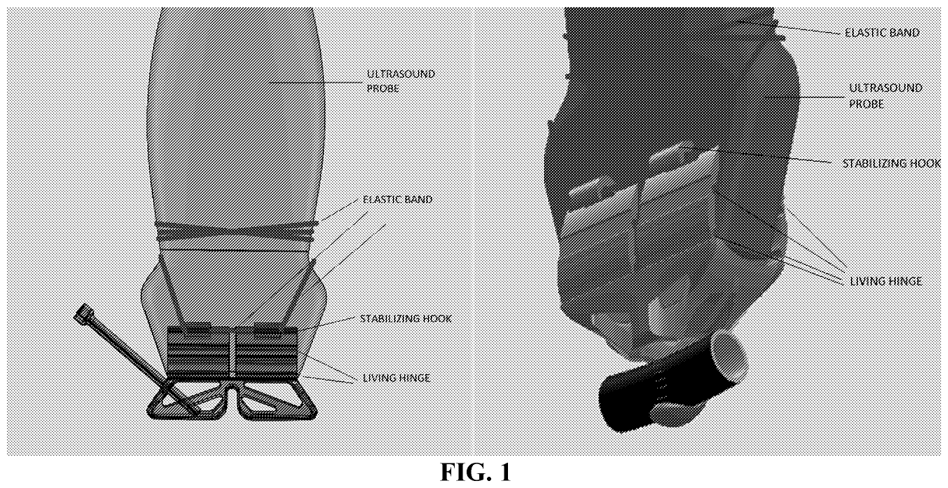

In one aspect, the present invention provides an ultrasound attachment for coupling an ultrasound probe (transducer) to an ultrasound coupling medium and securing the attachment to the probe. As illustrated in FIG. 1, an ultrasound probe is engaged with an ultrasound attachment described herein. The attachment receiving the probe with the living hinges on the attachment adjusting to attachment to particular contours of the probe. The attachment having stabilizing hooks that are configured to receive a band that is also engaged with the probe and any probe cover. FIG. 1 illustrates a probe having an attachment secured by the stabilizing hooks with a rubber band.

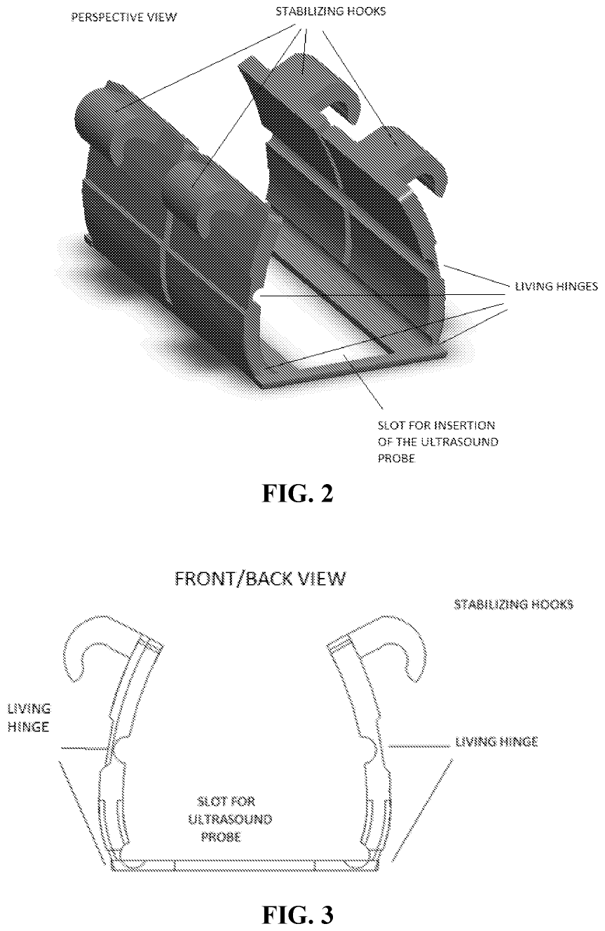

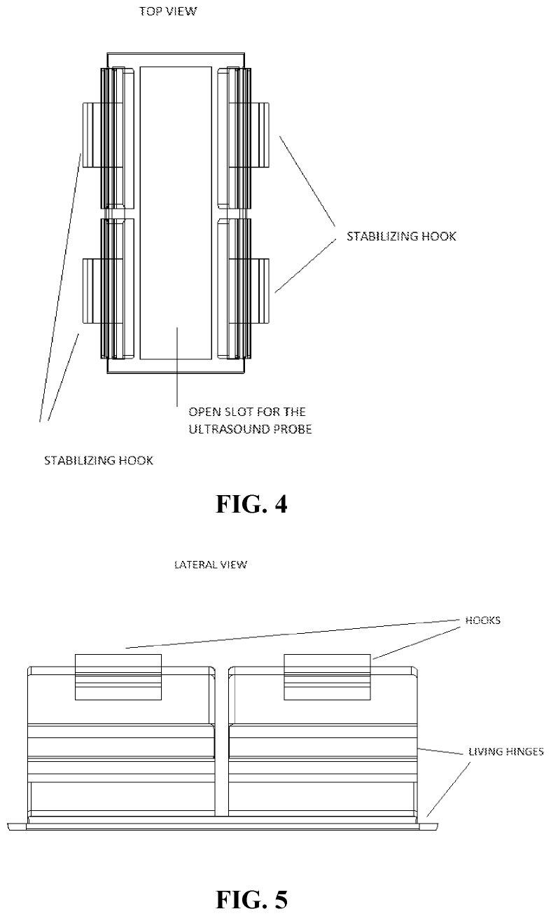

FIG. 2 is an illustration of a perspective view of one example of an attachment described herein. The attachment has a bottom configure for allowing operation of the ultrasound probe and two sides configured to receive and secure the probe, the sides having living hinges and stabilizing hooks. FIG. 3 is an end view of the attachment of FIG. 2. FIG. 4 is a top view of the attachment of FIG. 2. FIG. 5 is a side view of the attachment of FIG. 2.

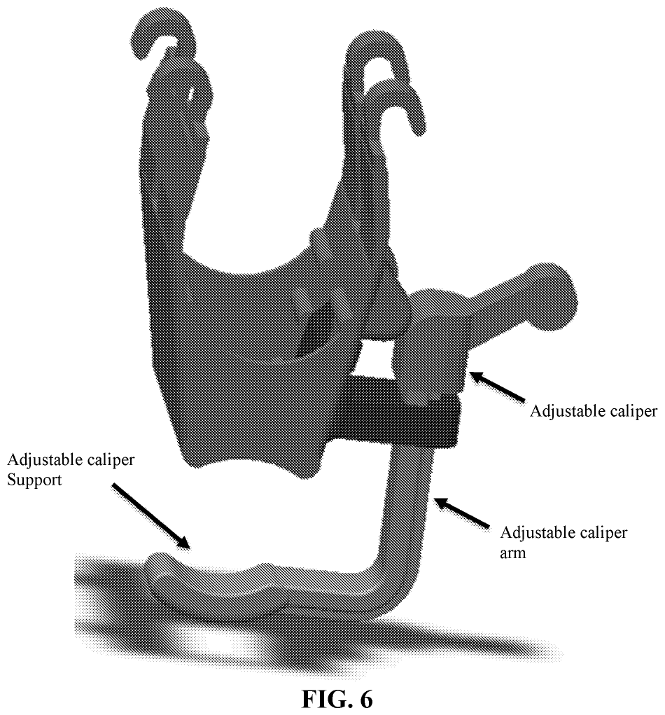

FIG. 6 illustrates an attachment with an adjustable caliper having an arm and a support that is configured for the distal portion to be placed under a vessel and to reversibly secure the attachment to a vessel for imaging or assessment. The support is positioned at the distal end of the attachment below the attachment slot. The adjustable caliper has an arm that can be moved vertically to adjust the distance between the support and the attachment. The adjustable caliper can be locked at a particular position during use. The proximal end of the adjustable caliper can terminate in a handle. The support can be configured with a concave surface for position vessels in the caliper.

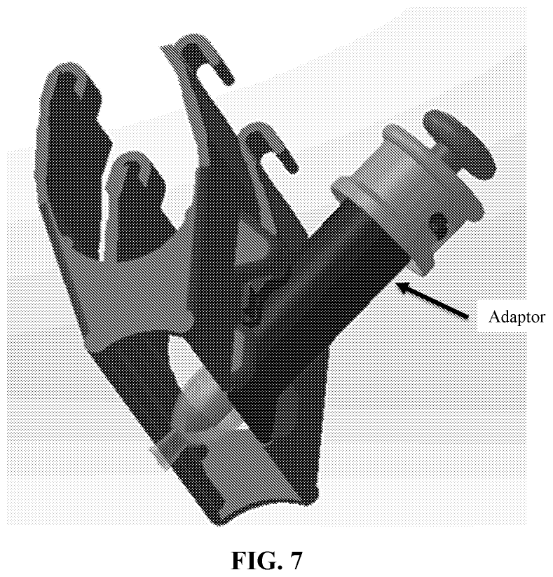

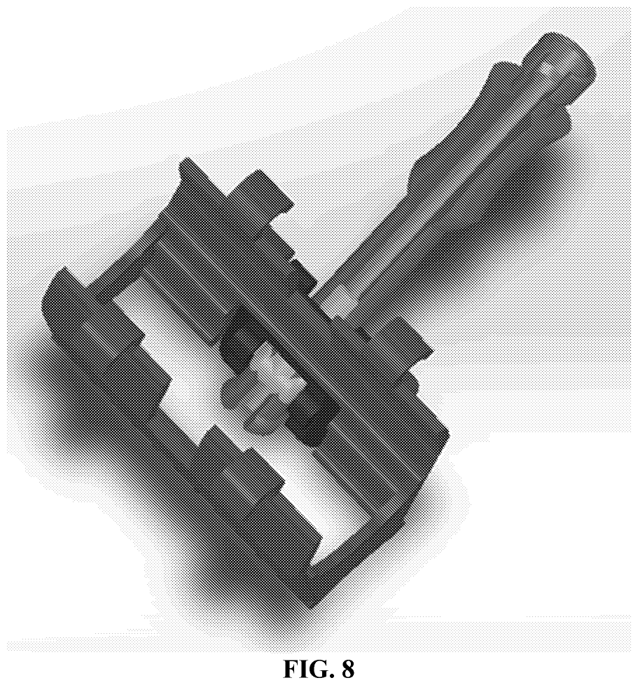

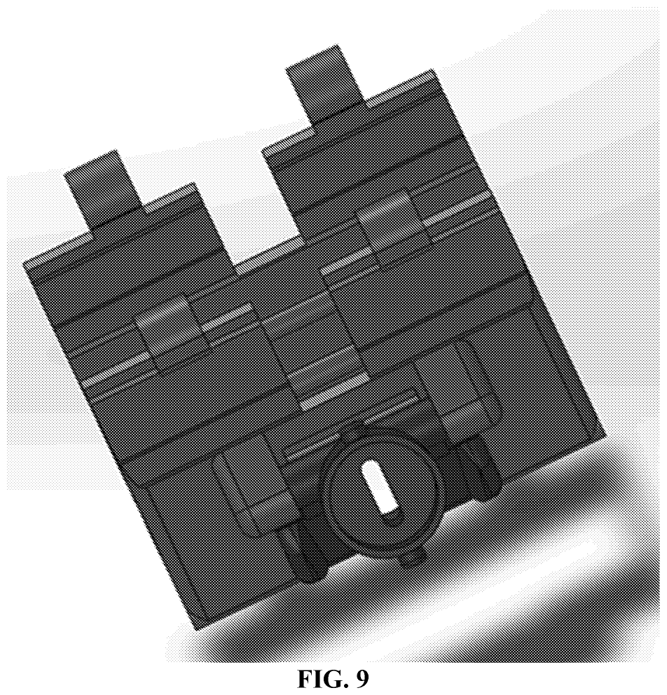

FIG. 7 to FIG. 9 illustrate one embodiment of the invention. The attachment system is used not only for needle guides but also support a ultrasound standoff adapter for intraoperative use. A standoff is comprised of different material with ideal ultrasound transmittance. The attachment system with an stand-off should be sterile for intra-operative used. A second attachment to the wall on the standoff chamber allows different functions such as; (1) adjustable caliper for vessel alignment and blood flow imaging and measurement in vascular anastomoses; (2) retractable Pen with biocompatible and sterile ink for making the surface of the organ based on internal imaging; and/or (3) plastic probing or adapter with hollowed introducer with different diameters; that allows insertion and real-time imaging of needles, electrocaltety, radio frequency ablation instruments, cryoablation systems, suture and vessel closure systems and stapling systems. The adapter can form a lumen that can be accessed from the proximal end of the adapter with the distal end position to provide an access point to the sonogram target area. Lumen of the adapter can have diameter of 1 mm to 3 cm in diameter, including all values and ranges there between. The adapter can be from 2 cm to 15 cm long and configured to for proving access for a pen or other medical instruments.

1. An ultrasound probe attachment comprising:
a bottom side configured to operably receive the transmission end of an ultrasound probe; and
a plurality of flexible side walls substantially perpendicular to and integrally formed with the bottom side to secure the probe.

2. The attachment of claim 1, wherein the side walls have living hinge portions integrally formed with the bottom side.

3. The attachment of claim 2, wherein the side walls have a plurality of stabilizing hooks configured to receive a flexible band attached to the probe.

4. The attachment of claim 1, further comprising an adjustable caliper.

5. The attachment of claim 1, further comprising an adapter.

6. An ultrasound probe attachment comprising:
a bottom side configured to operably receive the transmission end of an ultrasound probe; and
a plurality of flexible side walls substantially perpendicular to and integrally formed with the bottom side to secure the probe forming a standoff chamber, the standoff chamber configured to include a passage for an electrocauterizer.

7. The attachment of claim 6, wherein the standoff chamber is configured to permit alignment of the electrocautery with the ultrasound transducer.

8. The attachment of claim 6, further comprising an adjustable caliper.

9. The attachment of claim 6, further comprising an adapter.

10. A method comprising:
covering the transmission end of an ultrasound probe with an attachment comprising:
a bottom side configured to operably receive the transmission end of an ultrasound probe;
a plurality of flexible side walls having a plurality of stabilizing hooks, the side walls being substantially perpendicular to and integrally formed with the bottom side;
securing the attachment to the ultrasound probe using the stabilizing hooks to receive a flexible band attached to the probe;
moving the secured attachment in tandem with the ultrasound probe.
